(19) United States
(12) Patent Application Publication
(10) Pub. No.: US 2006/0025778 A1
(43) Pub. Date: Feb. 2, 2006

(54) Methods and apparatus for artificial disc replacement (ADR) insertion and other surgical procedures

(76) Inventor: Bret A. Ferree, Cincinnati, OH (US)

Correspondence Address: John G. Posa;Gifford, Krass, Groh, Sprinkle,, Anderson & Citkowski, P.C., PO Box 7021, Troy, MI, 48007, US

(21) Appl. No.: 11/184,480

(22) Filed: Jul. 19, 2005

Related U.S. Application Data
(60) Provisional application No. 60/589,752, filed on Jul. 21, 2004.

(51) Int. Cl. A61B 17/60

(52) U.S. Cl. 606/102

(57) ABSTRACT

Improved methods and apparatus render ADR insertion safer and more precise. Certain surgical instruments according to the invention include a level enabling a user to align the instrument for proper placement of a surgical implant. A different surgical instrument includes two or more scopes mounted relative to the instrument permitting a user to simultaneously view more than one side of the instrument. A device for use with a surgical instrument having a long shaft includes a holder that surrounds at least a portion of the shaft allowing a user to control the instrument with both hands. A further surgical instrument comprises a set of retractors and one or more guards placed over the retractors for protecting the great vessels, nerves or other delicate structures during a surgical procedure. Surgical apparatus according to the invention comprises a platform mountable to an operating room table over a patient undergoing a surgical procedure, enabling a user to place their hands or attach an instrument while operating. Different surgical instruments include an indicator showing angular displacement or a wedge-shaped portion used for disc distraction. Also disclosed are blades designed for use with a power tool featuring a cutting edge configured such that rapid oscillation of the cutting tool reduces the pressure a user must apply the tool.

### REFERENCE TO RELATED APPLICATION

This application claims priority from U.S. Provisional Patent Application Ser. No. 60/589,752, filed Jul. 21, 2004, the entire content of which is incorporated herein by reference.

### FIELD OF THE INVENTION

This invention relates generally to spine surgery and, in particular, to improved methods and apparatus for artificial disc replacement (ADR) insertion.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

Many spinal conditions, including degenerative disc disease, can be treated by spinal fusion or through artificial disc replacement (ADR). ADR has several advantages over spinal fusion. The most important advantage of ADR is the preservation of spinal motion. Spinal fusion eliminates motion across the fused segments of the spine. Consequently, the discs adjacent to the fused level are subjected to increased stress. The increased stress increases the changes of future surgery to treat the degeneration of the discs adjacent to the fusion.

ADRs move better and last longer when properly placed within the disc space. Prior art methods and apparatus for ADR insertion are modification of the devices and methods used to perform anterior interbody fusion. Fusion devices do not require precise placement. Improper placement of an ADR may result in markedly decreased range of Motion (ROM) permitted by the ADR. Furthermore, improperly placed ADRs may wear quickly or loosen from the Vertebral Endplates (VEPs).

### SUMMARY OF THE INVENTION

This invention improves upon prior art methods and apparatus by making ADR insertion safer and more precise. Certain surgical instruments according to the invention include a level enabling a user to align the instrument for proper placement of a surgical implant. The implant may be an artificial disc replacement (ADR) or form parts of a total hip or other joint replacement.

A different surgical instrument includes two or more scopes mounted relative to the instrument permitting a user to simultaneously view more than one side of the instrument. The instrument may comprise a set of retractors.

A device for use with a surgical instrument having a long shaft includes a holder that surrounds at least a portion of the shaft allowing a user to control the instrument with both hands.

A further surgical instrument comprises a set of retractors and one or more guards placed over the retractors for protecting the great vessels, nerves or other delicate structures during a surgical procedure. Differently shaped guards may be provided for different spinal levels.

Surgical apparatus according to the invention comprises a platform mountable to an operating room table over a patient undergoing a surgical procedure, enabling a user to place their hands or attach an instrument while operating. The instrument may be a retractor, a guide to control the movement of a tool, of other instruments.

Different surgical instruments include an indicator showing angular displacement or a wedge-shaped portion used for disc distraction. Also disclosed are blades designed for use with a power tool featuring a cutting edge configured such that rapid oscillation of the cutting tool reduces the pressure a user must apply the tool.

### BRIEF DESCRIPTION OF THE INVENTION

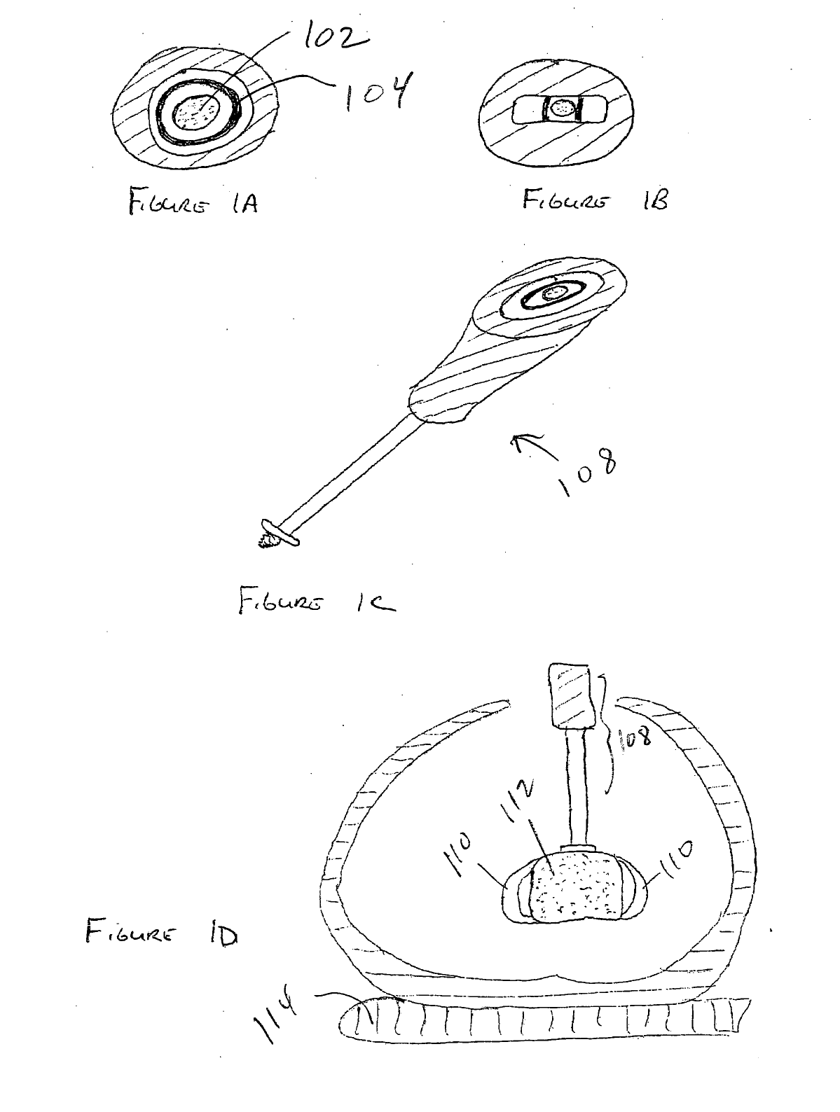

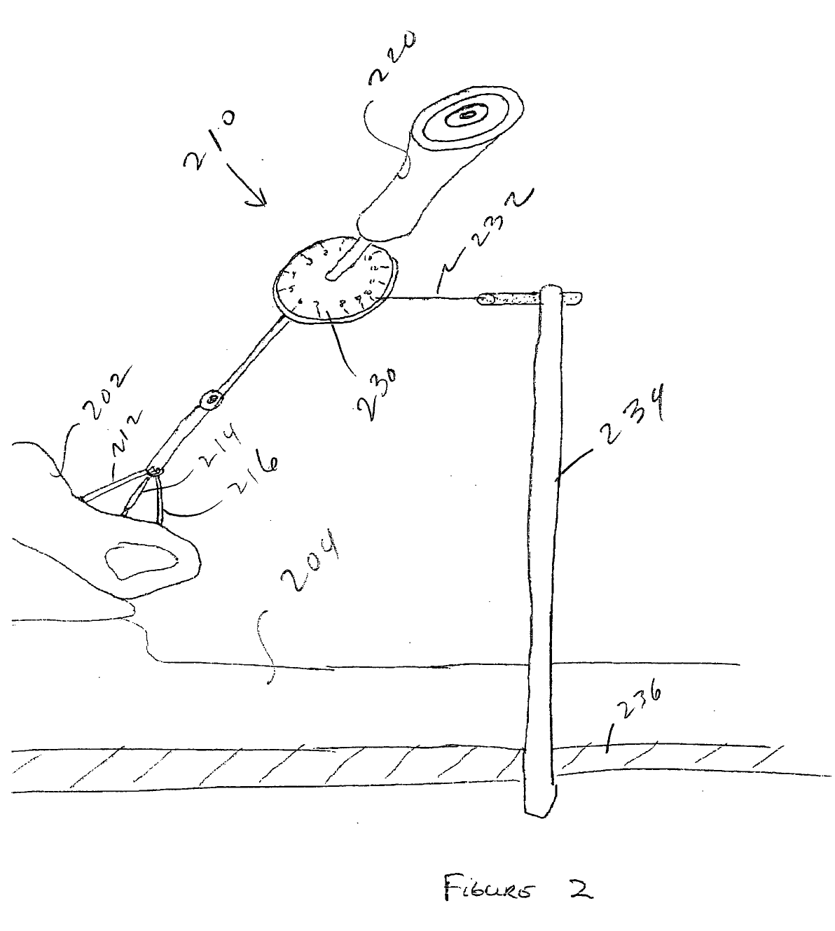

FIG. 1A is a view of the end of the handle of a surgical instrument according to the present invention;

FIG. 1B is a view of the end of the handle of a surgical instrument with an alternative embodiment of the level;

FIG. 1C is an oblique view of a surgical instrument and the embodiment of the level drawn in FIG. 1A;

FIG. 1D is an axial cross section of the body, a disc, an ADR, an operating room table, and the instrument drawn in FIG. 1C;

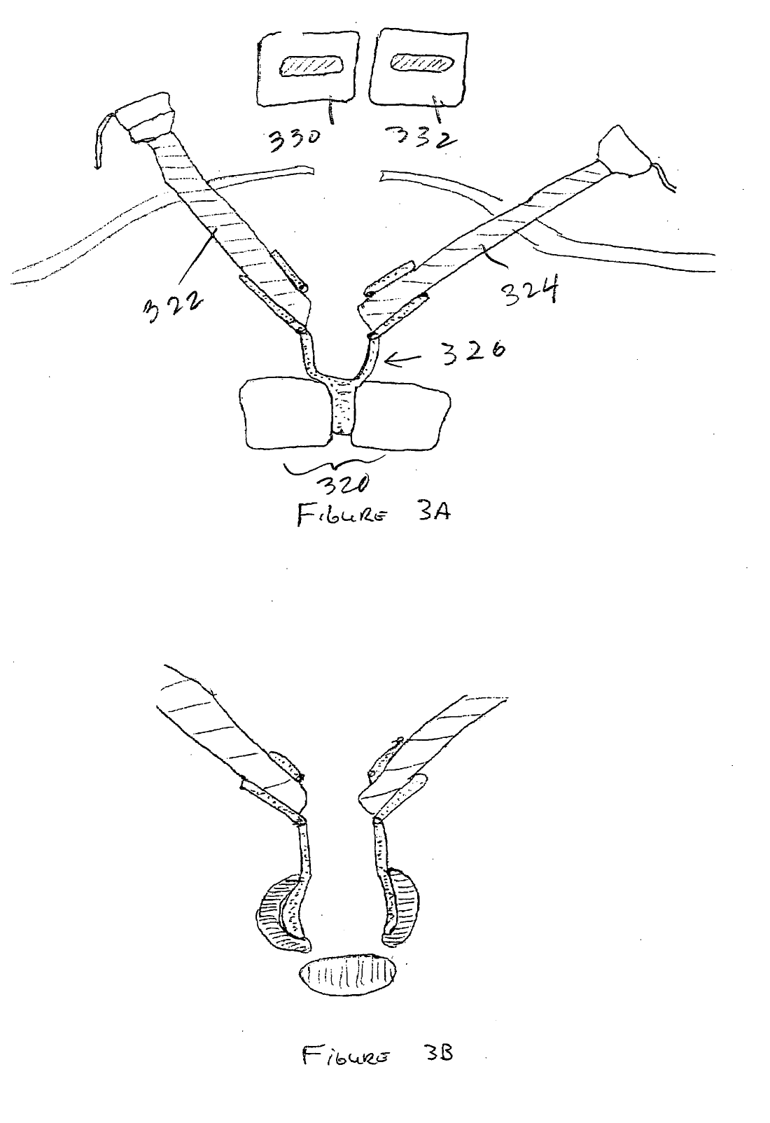

FIG. 2 is a posterior view of the pelvis, the left lower extremity, and an oblique view of an alternative embodiment of a device used to properly align the cup of a total hip replacement (THR);

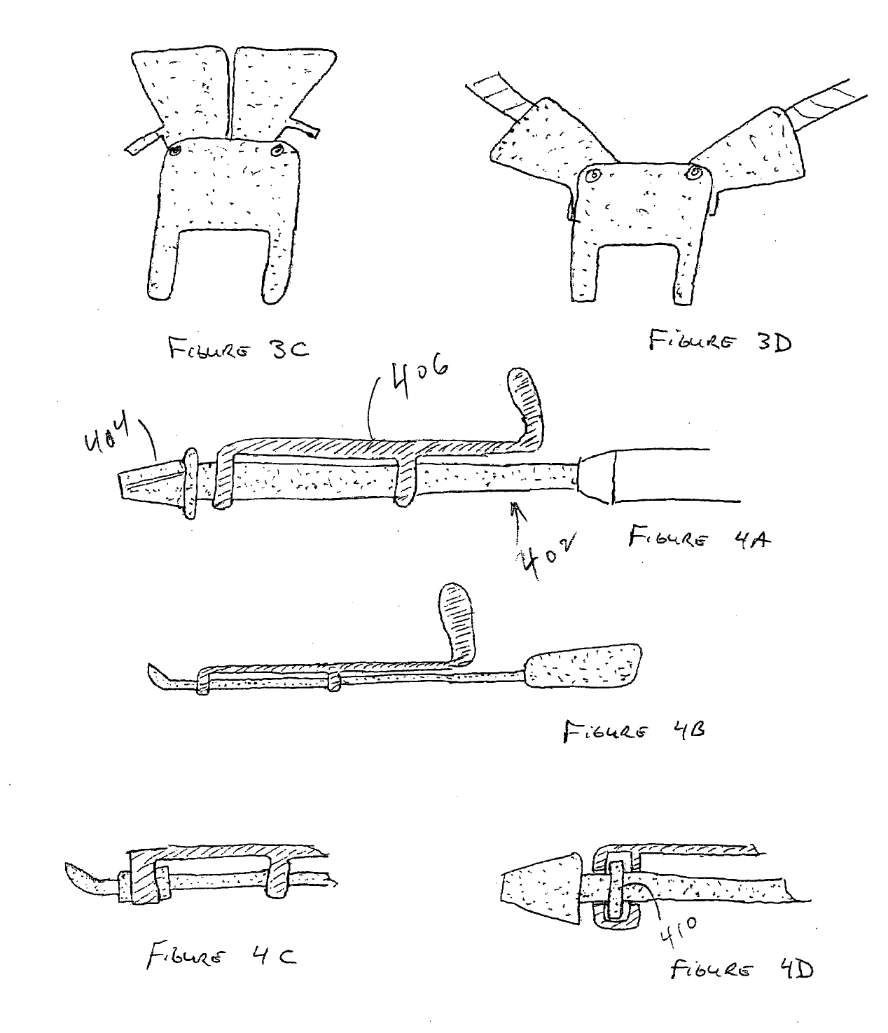

FIG. 3A is a partial sagittal cross section of the spine, two scopes, a retraction device, and two monitors;

FIG. 3B is an axial cross section of an alternative embodiment of the device drawn in FIG. 3A and a disc;

FIG. 3C is a lateral view of the retractor drawn in FIG. 3A;

FIG. 3D is a lateral view of the retractor drawn in FIG. 3C and two scopes;

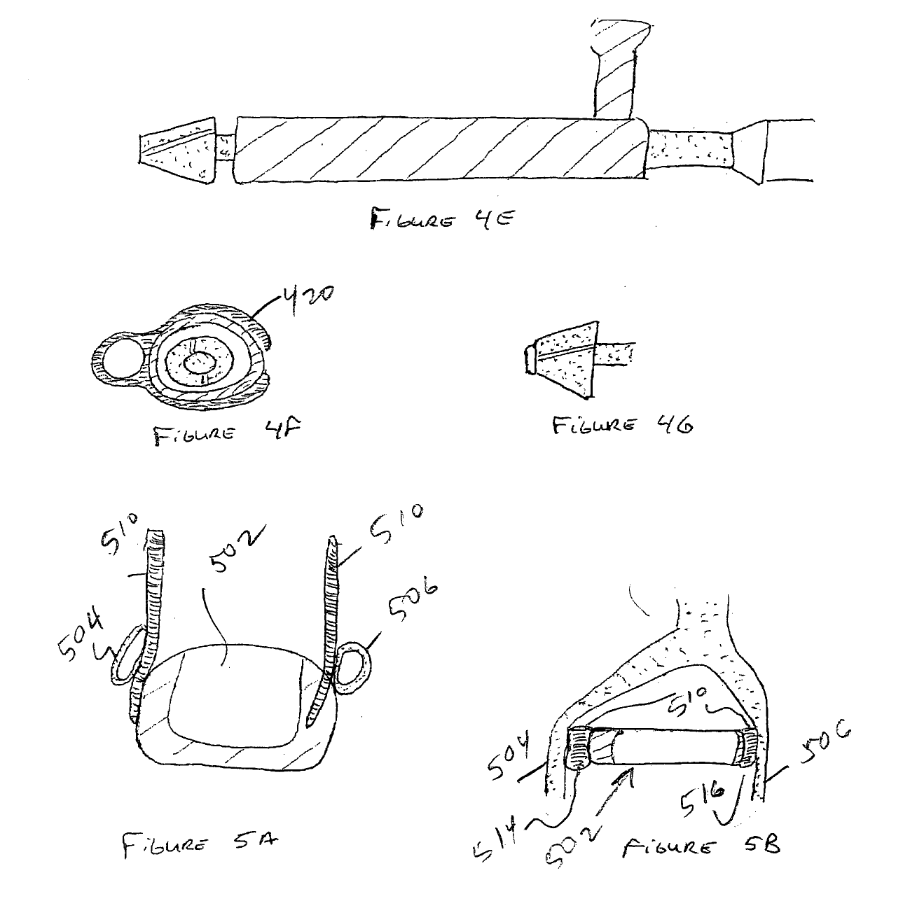

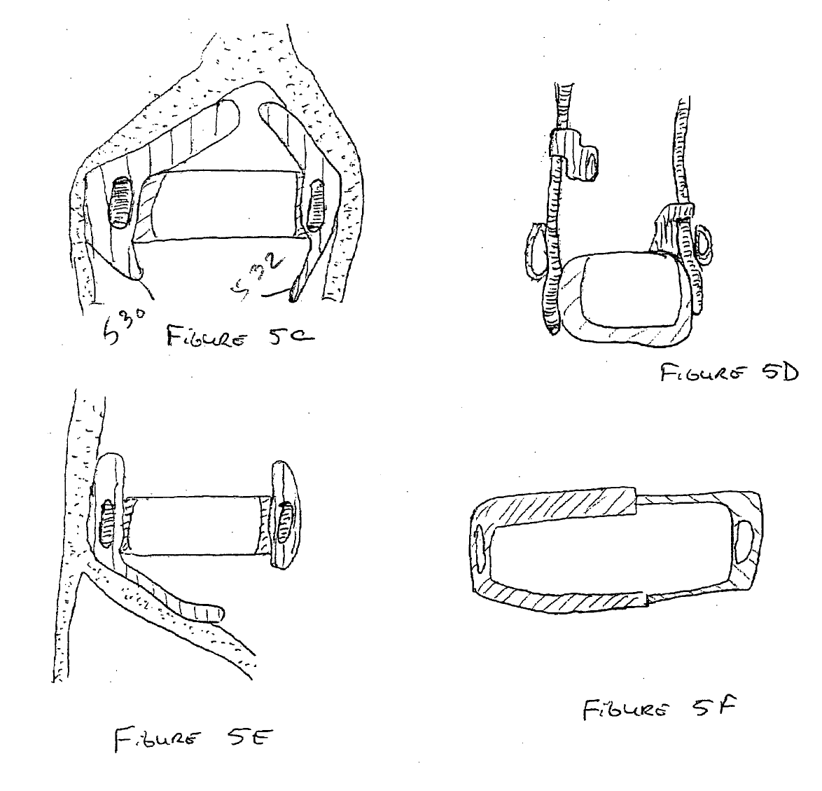

FIG. 4A is lateral view of a power tool, a reamer bit, and a holding instrument according to the present invention;

FIG. 4B is lateral view of the holding tool drawn in FIG. 4A and a curette;

FIG. 4C is a lateral view of a lateral view of the tip of an alternative embodiment of the holding tool drawn in FIG. 4B and a curette;

FIG. 4D is a cross section of an alternative embodiment of the present invention;

FIG. 4E is a lateral view of an alternative embodiment of the holding tool drawn in FIG. 4D, a reamer bit, and a power tool;

FIG. 4F is a view of the end of the holding device drawn in FIG. 4E, a reamer bit, and an attachment that may house a suction device, a scope, and/or a light;

FIG. 4G is a lateral view of the tip of an alternative reamer bit which is blunt;

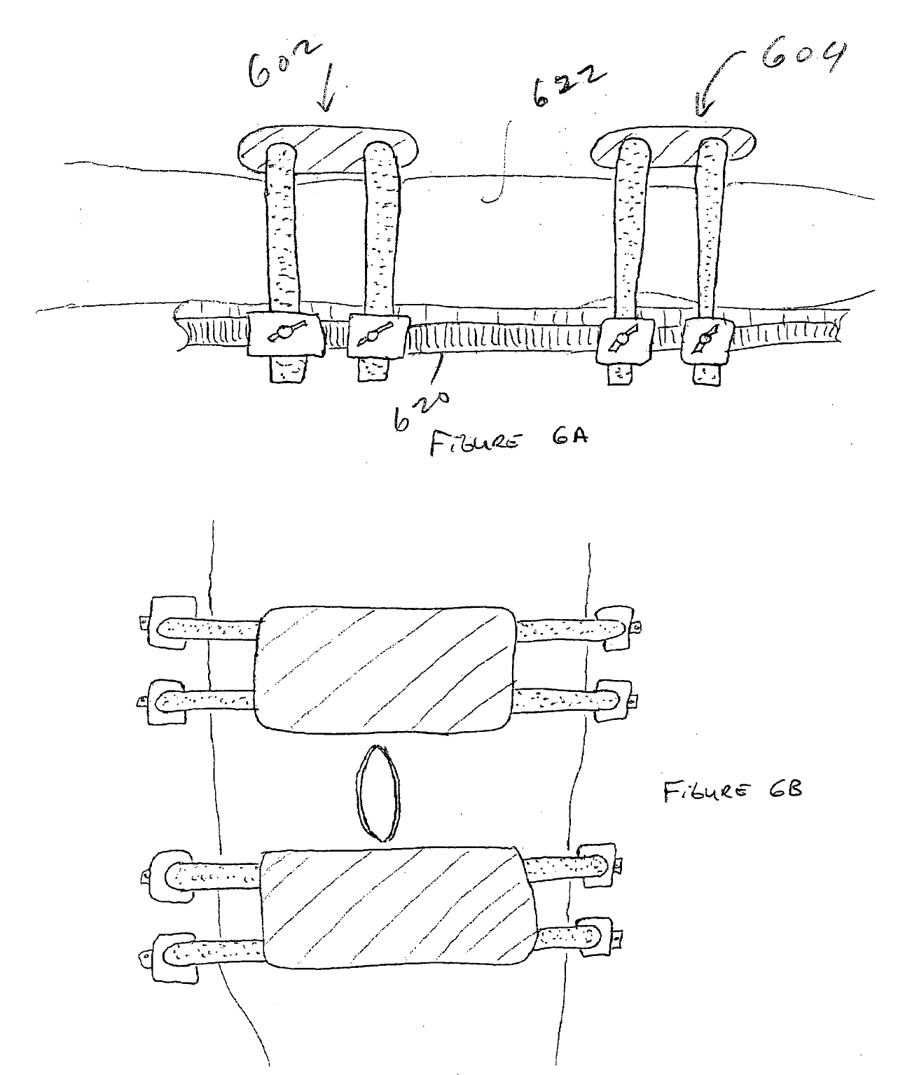

FIG. 5A is an axial cross section of a disc, two great vessels, and novel retractor;

FIG. 5B is an anterior view of the disc, the great vessels, and the embodiment of the retractor drawn in FIG. 5A;

FIG. 5C is an anterior view of the disc, the great vessels, the embodiment of the retractors drawn in FIG. 5B;

FIG. 5D is an axial cross section of the disc, the great vessels, and the retractor drawn in FIG. 5C;

FIG. 5E is an anterior view of the disc, the great vessels, the retractors drawn in FIG. 5C;

FIG. 5F is a view of the top of an alternative embodiment of the guard drawn in FIG. 5C;

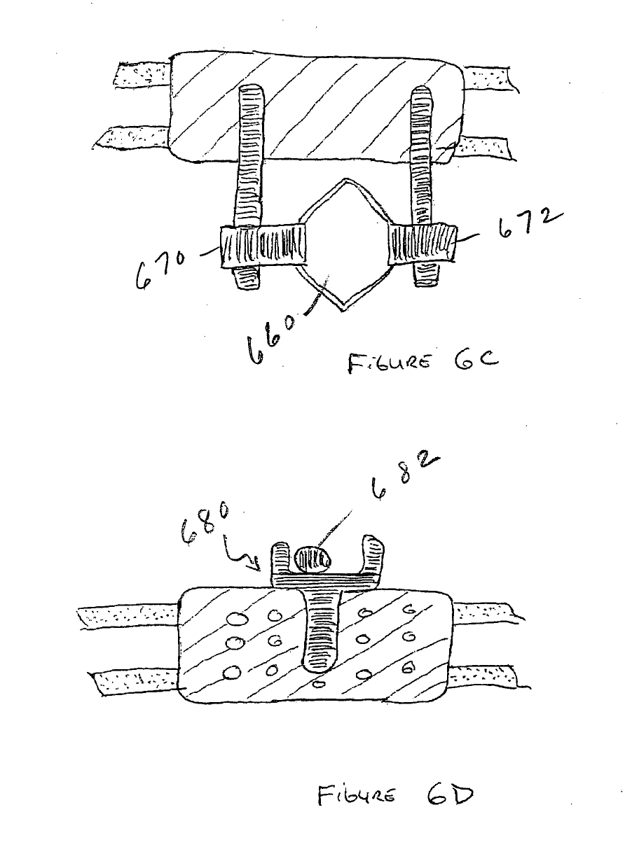

FIG. 6A is a lateral view of novel platforms, an operating room table, and a patient;

FIG. 6B is view of the anterior side of the platforms drawn in FIG. 6A, and a patient;

FIG. 6C is an anterior view of the platform drawn in FIG. 6B and a surgical wound;

FIG. 6D is an anterior view of the platform drawn in FIG. 6B and a novel guide attached to the platform;

FIG. 6E is an anterior view of the platform drawn in FIG. 6D and an alternative guide that controls the depth the instrument extends into the disc;

FIG. 6F is a lateral view of the platform drawn in FIG. 6B, a guide, and a reamer;

FIG. 6G is an anterior view of the platform drawn in FIG. 6B;

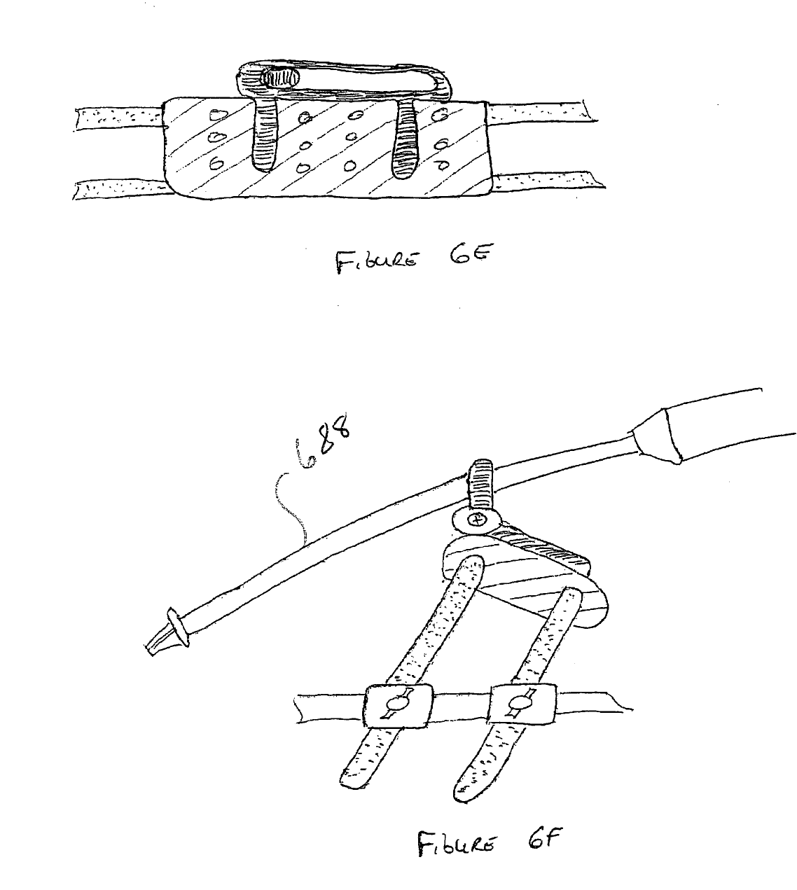

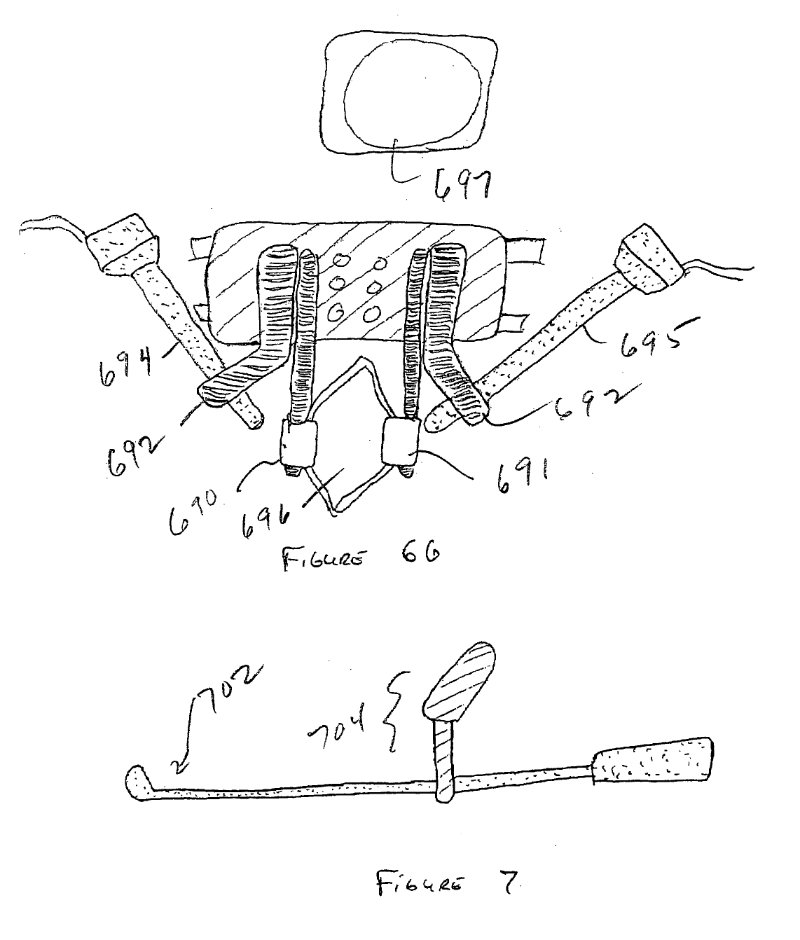

FIG. 7 is a lateral view of a curette and a handle that is reversibly attached to the shaft of this or other surgical instruments;

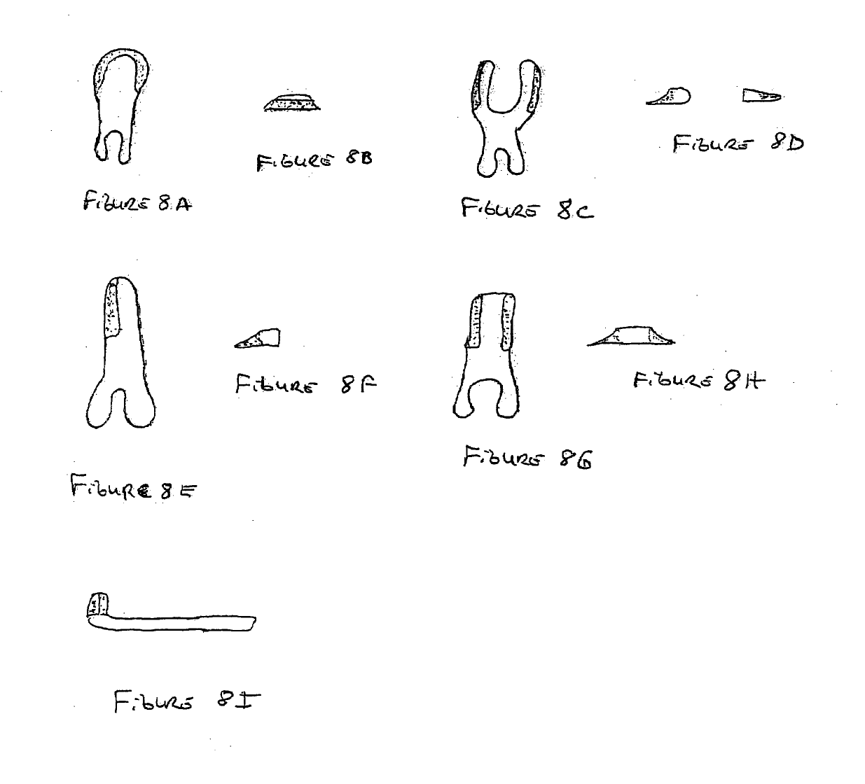

FIG. 8A is the view of the top of a blade designed for use with a power tool;

FIG. 8B is a view of the end of the cutting tool drawn in FIG. 8A;

FIG. 8C is a view of the top of an alternative cutting bit;

FIG. 8E is a view of the top of an alternative cutting bit;

FIG. 8F is a view of the end of the cutting tool drawn in FIG. 8E;

FIG. 8G is a view of the top of an alternative cutting bit;

FIG. 8H is a view of the end of the cutting tool drawn in FIG. 8G;

FIG. 8I is a view of the side of an alternative cutting tool;

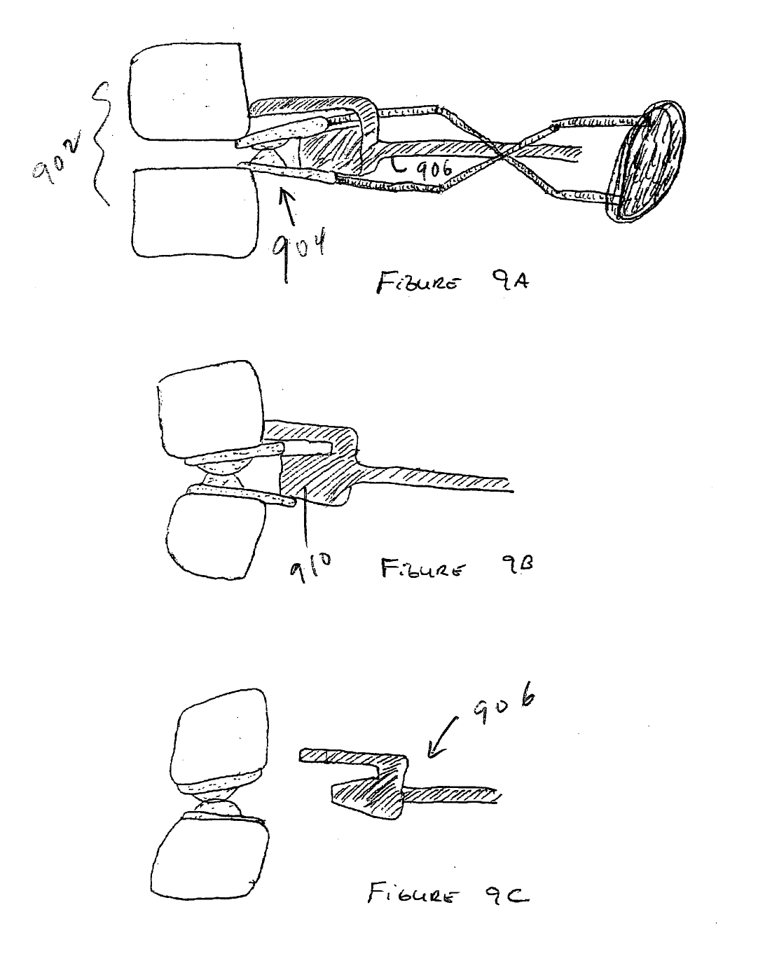

FIG. 9A is a lateral view of the spine, an ADR, and an insertion tool;

FIG. 9B is a lateral view of the spine, an ADR and the wedge tool drawn in FIG. 9A;

FIG. 9C is a lateral view of the spine, an ADR and the wedge tool drawn in FIG. 9B;

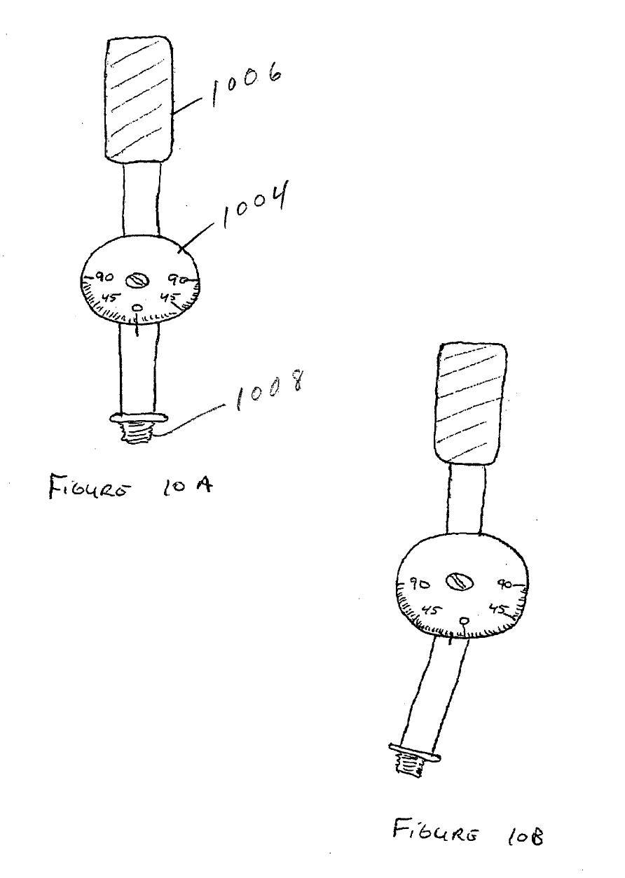

FIG. 10A is a drawing of a tool according to the present invention; and

FIG. 10B shows the tool at and angle off center.

### DETAILED DESCRIPTION OF THE INVENTION

FIG. 1A is a view of the end of the handle of a surgical instrument according to the invention incorporating a bubble level. The dotted circle 102 in the center of the drawing represents a gas bubble. The dark ring 104 outside the bubble represents the target for the bubble. The level helps the surgeon align his instrument.

FIG. 1B is a view of the end of the handle of a surgical instrument with an alternative embodiment of the level. FIG. 1C is an oblique view of a surgical instrument 108 and the embodiment of the level drawn in FIG. 1A. FIG. 1D is an axial cross section of the body, a disc 110, an ADR 112, an operating room (OR) table 114, and the instrument 108 drawn in FIG. 1C. Using the level, the surgeon can assure his instrument, and the attached ADR, are perpendicular to the OR table. Thus, as long as the patient is lying properly on the OR table, and the patient does not have a rotational abnormality of the spine, the novel instrument assures the ADR is placed with the proper rotational alignment.

FIG. 2 is a posterior view of the pelvis 202, the left lower extremity 204, and an oblique view of an alternative embodiment of a device 210 used to properly align the cup of a Total Hip Replacement (THR). Three legs 212, 214, 216 from the device are placed around the patient's acetabulum. The handle 220 of the instrument incorporates the level drawn in FIG. 1A. The surgeon tightens the connector between the shaft of the instrument and the portion of the instrument that holds the three legs of the device, after the bubble level is centered. The surgeon also records a reading from a circular guide 230 on the instrument. The reading is obtained by recording where a laser light 232 is pointing on the dial. The laser light is attached to a holding device 234 that is attached to the OR table 236. The novel invention records the alignment of a patient's acetabulum. The surgeon may use the device and the readings recorded from the device to properly align a prosthetic cup of a THR. The prosthetic cup is aligned properly when the legs of the device contact the cup, the handle is level, and the light shines on the proper number on the guide. Novel levels can be used on other surgical instruments.

FIG. 3A is a partial sagittal cross section of the spine 320, two scopes 322, 324, a retraction device 326, and two monitors 330, 332. The retraction device 326 is impacted into the disc, and the scopes are held in openings in the retractor. The scopes may be placed through small, separate, incisions. Cameras transmit data from the scopes to the monitors. The scopes also transmit light into the retractor. The invention improves upon the use of surgical loupes and the operating microscope. A surgeon's hands, the handles of the instruments, the shafts of the instruments, and power tools attached to the instrument may obstruct the view provided by surgical loupes and surgical microscopes. This aspect of the invention places the tip of the scopes distal to the object that obstructs the view. Use of two or more scopes permits surgeons to see on more than one side of the instrument simultaneously. The improved visualization provide by multiple scopes decrease the risk of injuring structures on the “blind side” of a instrument that obstructs the view provided by a single scope. Surgeons may observe more than one monitor. Alternatively, the data to a single monitor could be changed.

Voice activated controls could be used to have the view provided by the monitors switch from the “left” scope to the “right” scope by saying “right”. If more than two scopes are used the surgeon could say “one” to refer to the scope at the one o'clock position, “four” for the scope at the four o'clock position etc. Multiple scopes may be placed into other holding devices for other types of surgery. For example, multiple scopes could be placed into a C-shaped ring like device for general surgical procedures, cardiac procedures, and other types of surgical procedures.

FIG. 3B is an axial cross section of an alternative embodiment of the device drawn in FIG. 3A and a disc. The scopes extend through the left and right sides of the retractor. FIG. 3C is a lateral view of the retractor drawn in FIG. 3A. The retractor is drawn in its collapsed shape. The collapsed shape facilitates insertion of the retractor through small incisions. FIG. 3D is a lateral view of the retractor drawn in FIG. 3C and two scopes. The retractor is drawn in its open position.

FIG. 4A is lateral view of a power tool 402, a reamer bit 404, and a holding instrument 406 according to the invention. Prior art instruments are difficult to control. The instruments have long shafts to extend from a patient's spine to beyond the surface of the abdomen. Surgeons control the prior art instrument by holding a handle that is several inches from the working portion of the tool. The instrument shown in FIG. 4 allows surgeons to control instruments with both of their hands. The novel instrument also allows surgeons to manipulate the tool closer to the working end of the tool.

FIG. 4B is lateral view of the holding tool drawn in FIG. 4A and a curette. The novel holding tool may be used with other surgical instruments. FIG. 4C is a lateral view of a lateral view of the tip of an alternative embodiment of the holding tool drawn in FIG. 4B and a curette. Enlargements on the shaft of the curette and the holding tool cooperate to improve the control of the instrument.

FIG. 4D is a cross section of an alternative embodiment of the invention, wherein an enlargement 410 on the shaft of the reamer is contained within an enlarged area in the holding tool. FIG. 4E is a lateral view of an alternative embodiment of the holding tool drawn in FIG. 4D, a reamer bit, and a power tool. The holding tool drawn in FIG. 4D incorporates a sleeve. The sleeve protects the soft tissues from the rotating shaft of the reamer bit. The handle of the holding device is represented by the projection from the top of the device. FIG. 4F is a view of the end of the holding device drawn in FIG. 4E, a reamer bit, and an attachment 420 that may house a suction device, a scope, and/or a light. FIG. 4G is a lateral view of the tip of an alternative reamer bit which is blunt.

FIG. 5A is an axial cross section of a disc 502, two great vessels 504, 506 and novel retractors 510. The retractors may extend into the disc or outside the disc. The retractors hold the great vessel away from the anterior portion of the disc. FIG. 5B is an anterior view of the disc 502, the great vessels 504, 506, and the embodiment of the retractor drawn in FIG. 5A. The retractors are holding the great vessels off of the anterior surface of the disc. FIG. 5C is an anterior view of the disc, the great vessels, the embodiment of the retractors drawn in FIG. 5B and novel guards 514, 516 that are placed over the retractors. The modular guards lie along the inner sides of the great vessels. Guards of other sizes and other shapes may be used.

The guards 530, 532 drawn in FIG. 5C are particularly suited to protect the great vessels lying adjacent to the L5/S1 disc. FIG. 5D is an axial cross section of the disc, the great vessels, and the retractor drawn in FIG. 5C. The guard over the retractor on the right side of the drawing has been placed against the disc. The guard on the retractor drawn on the left side of the drawing has not been lowered to the disc yet. FIG. 5E is an anterior view of the disc, the great vessels, the retractors drawn in FIG. 5C, and guards with alternative shapes to the shapes drawn in FIG. 5C. The shape of the guards facilitates retraction of the great vessels at the L4/L5 disc. FIG. 5F is a view of the top of an alternative embodiment of the guard drawn in FIG. 5C. The telescoping guard fits over the retractors drawn in FIG. 5C.

FIG. 6A is a lateral view of novel platforms 602, 604, an OR table 620, and a patient 622. The novel platforms lie over the patient. The platforms are attached to the side of the OR table. FIG. 6B is view of the anterior side of the platforms drawn in FIG. 6A, and a patient. The patient is lying in a supine position. The platforms may be seen cephalad and caudal to an incision. Surgeons may rest their hands on the platforms. Much like a tripod used with guns or cameras, the platforms help surgeons avid undesired movements of their instruments. Instruments with long shafts are particularly difficult to control. Surgeons may also lever instruments against the stable platforms. Surgeons are not able to lever their instruments against the soft tissues of the abdomen.

FIG. 6C is an anterior view of the platform drawn in FIG. 6B and a surgical wound 660. Retractors 670, 672 can be seen extending from the platform to the surgical wound. FIG. 6D is an anterior view of the platform drawn in FIG. 6B and a novel guide 680 attached to the platform. The guide is used to control an instrument. The circle 682 represents the cross section of an instrument. The guide limits the movements of the instrument. The instrument and the guide have features that control the depth the instrument is inserted into the wound, the excursion allowed by the instrument from the left to the right.

For example, the platform and guide may be used with the reamer drawn in FIG. 4E. The guide controls the depth the reamer extends into the disc and how far the reamer may be directed to the left or right. FIG. 6E is an anterior view of the platform drawn in FIG. 6D and an alternative guide that controls the depth the instrument extends into the disc, how far the instrument may be moved to the left and the right, and how far the instrument may be moved towards the head and towards the feet. FIG. 6F is a lateral view of the platform drawn in FIG. 6B, a guide, and a reamer 688. The platform and the guide are fixed in angles that facilitate use of the instrument. The platform and the guide may be fixed at other angles. This embodiment of the device facilitates use of instruments in the L5/S1 disc. The L5/S1 disc space is generally tilted forward with respect to the patient's other discs. This embodiment of the invention also facilitates use of fluoroscopy during the procedure. The platforms, guides, and retractors are preferably made of radiolucent material.

FIG. 6G is an anterior view of the platform drawn in FIG. 6B, retractors 690, 691, scope holders 692, 693, two scopes 694, 695, a surgical wound 696 and a monitor 697. The scopes are reversibly held by attachments from the platforms. The scopes may be placed through separate surgical incisions. The platform holds the scopes more steadily than surgical assistants. In fact the platform may eliminate the need for surgical assistants for several types of surgical procedures.

FIG. 7 is a lateral view of a curette 702 and a handle 704 that is reversibly attached to the shaft of this or other surgical instruments. The handle gives surgeons more control of their instruments.

FIG. 8A is the view of the top of a blade designed for use with a power tool. For example, the blade could be attached to an oscillating power tool. The cutting edge of the tool is represented by the dotted area of the drawing. Rapid oscillation of the cutting tool reduces the pressure surgeons must apply the tool. Prior art, non-power instruments such as curettes and elevators require a great deal of pressure to cut or separate the tissues. The reduced pressure required to operate the power tools decreases the risk of an instrument slipping if the resistance provided by the soft tissues drops suddenly.

FIG. 8B is a view of the end of the cutting tool drawn in FIG. 8A. FIG. 8C is a view of the top of an alternative cutting bit. The bit has cutting surfaces along the sides of the bit. FIG. 8D is a view of the end of the cutting tool drawn in FIG. 8C. FIG. 8E is a view of the top of an alternative cutting bit. The bit has a cutting surface along one side of the bit. FIG. 8F is a view of the end of the cutting tool drawn in FIG. 8E. FIG. 8G is a view of the top of an alternative cutting bit. FIG. 8H is a view of the end of the cutting tool drawn in FIG. 8G. FIG. 8I is a view of the side of an alternative cutting tool. The cutting portion of the bit is at angle to the shaft of the tool.

FIG. 9A is a lateral view of the spine 902, an ADR 904, and an ADR insertion tool 906. A novel, wedge-shaped portion 910 of the tool holds the TDR in an exaggerated trapezoid shape. The trapezoid shape of the ADR facilitates insertion of the ADR into the prepared disc space. A portion of the wedge-shaped tool extends onto the vertebra or both vertebrae. The ADR is impacted into the disc space. The ADR moves off of the wedge as it moves into the disc space. The ADR assumes a less trapezoid shape as it enters the disc space. The invention enables the ADR to distract the disc space as the ADR is inserted into the disc space. The tool that holds the ADR may have scissor arm components. FIG. 9B is a lateral view of the spine, an ADR and the wedge tool drawn in FIG. 9A. The ADR has been partially inserted into the disc space. The portion of the tool that rests against the vertebra or vertebrae prevents the wedge tool from entering the disc space with the ADR. The tool used to impact the ADR was not drawn to show the wedge tool more clearly. FIG. 9C is a lateral view of the spine, an ADR and the wedge tool drawn in FIG. 9B. The ADR is positioned within the disc space. The ADR was driven off of the wedge tool as it was impacted into the disc space.

FIG. 10A is a drawing of a tool according to the invention which is articulated through a dial 1004 showing the degree of angulation between the handle 1006 and a shaft having a threaded tip 1008. FIG. 10B shows the tool at and angle off center. The threaded tip 1008 may be connected to an ADR or other implant, with the dial being used to indicate off-axis placement. If on-axis placement is preferred, the tool may be manipulated until a “zero” reading occurs. Alternatively, if an off-axis manipulation is indicated, the surgeon need only read the value off of the dial on either side of center.

I claim:

1. A surgical instrument, comprising:
a level enabling a user to orient the instrument in space for proper placement of a surgical implant.

2. The instrument of claim 1, wherein the implant is an artificial disc replacement (ADR).

3. The instrument of claim 1, wherein the implant forms parts of a total joint replacement.

4. A surgical instrument, comprising:
two or more viewing scopes mounted relative to the instrument permitting a user to simultaneously view more than one side of the instrument.

5. The surgical instrument of claim 4, wherein the instrument comprises a set of retractors.

6. A device for use with a surgical instrument having a long shaft, comprising:
a holder that surrounds at least a portion of the shaft allowing a user to control the instrument with both hands.

7. A surgical instrument, comprising:
a set of retractors; and
one or more guards placed over the retractors for protecting the great vessels, nerves or other delicate structures during a surgical procedure.

8. The surgical instrument of claim 7, including differently shaped guards for different spinal levels.

9. Surgical apparatus, comprising:
a platform mountable to an operating room table over a patient undergoing a surgical procedure, enabling a user to place their hands or attach an instrument while operating.

10. The surgical apparatus of claim 9, wherein the instrument is a retractor.

11. The surgical apparatus of claim 9, wherein the instrument is a guide to control the movement of a tool.

12. A surgical instrument, comprising:
an indicator showing angular displacement.

13. A surgical instrument, comprising:
a wedge-shaped portion used for disc distraction.

14. A blade designed for use with a power tool, comprising:
a cutting edge configured such that rapid oscillation of the cutting tool reduces the pressure a user must apply the tool.
